(19) United States
(12) Patent Application Publication
(10) Pub. No.: US 2006/0217350 A1
(43) Pub. Date: Sep. 28, 2006

(54) Disodium pamidronate formulation

(75) Inventors: Dhiraj Khattar, Sahibabad (IN); Manish Grover, Sahibabad (IN); Mukesh Kumar, Sahibabad (IN); Prasanna Kumar Srinivasa Reddy Gari Jonnala, Sahibabad (IN); Shobhit Singh, Sahibabad (IN); Rama Mukherjee, Sahibabad (IN); Anand C. Burman, Sahibabad (IN)

Correspondence Address: HAMRE, SCHUMANN, MUELLER & LARSON, P.C., P.O. BOX 2902-0902, MINNEAPOLIS, MN, 55402, US

(73) Assignee: DABUR PHARMA LTD., New Delhi (IN)

(21) Appl. No.: 11/245,389

(22) Filed: Oct. 6, 2005

(30) Foreign Application Priority Data
Mar. 24, 2005 (IN) 228/KOL/2005

(51) Int. Cl. A61K 31/66

(52) U.S. Cl. 514/102

(57) ABSTRACT

A stable pharmaceutical composition comprising a solution of disodium pamidronate in an aqueous solvent and a process for the preparation thereof. The solution of disodium pamidronate is free of particulate matter and has an alkaline pH. The process for preparation of the stable pharmaceutical composition comprises adding disodium pamidronate and optionally a sugar into an aqueous solvent; heating the said mixture at a temperature in the range of 50° C. to 90° C. to obtain a clear solution. It is cooled to ambient temperature, filtered, filled and stored in a conventional and untreated glass container and sealed with normal elastomeric closure. The sealed container is sterilized by steam sterilization.

### FIELD OF THE INVENTION

The present invention relates to a stable pharmaceutical composition of pamidronate disodium and to a process for the preparation thereof.

### BACKGROUND OF THE INVENTION

Bisphosphonates are potent inhibitors of osteoclast-mediated bone resorption and are effective in the treatment of malignant bone disease. Intravenous bisphosphonates are the current standard of care for the treatment of hypercalcemia of malignancy (HCM) and for the prevention of skeletal complications associated with bone metastases.

Currently, disodium pamidronate in a crystalline pentahydrate form (U.S. Pat. No. 4,711,880) and zoledronic acid (U.S. Pat. No. 4,939,130) are the only agents recommended by the American Society of Clinical Oncology (ASCO) for the treatment of bone lesions from breast cancer and multiple myeloma. Furthermore, disodium pamidronate is approved by US FDA for the treatment of Paget's disease, osteolytic bone metastases of breast cancer and multiple myeloma in conjunction with standard antineoplastic therapy.

Currently, pamidronate disodium is marketed both as a lyophilized injection formulation and a ready to use injectable solution. The former mode of administration is, however, associated with several disadvantages such as:
a) Double handling: To administer a lyophilized preparation, double handling of the drug is required. The lyophilized cake has to be first reconstituted with water for injection, then diluted with the infusion fluid and then administered to the patient;
b) Dissolution time of the cake: In some cases, the complete dissolution of the powder may require prolonged shaking because of solubilisation problems;
c) Health Hazard: Improper reconstitution of a lyophilized powder sometimes result in the formation of air-borne droplets (“blow-back”), which, in the case of a potent calcium regulator such as bisphosphonates may be a health hazard to the personnel making up the solution for injection;
d) Improper dose: There is always a problem in reconstituting a lyophilized powder in that an inappropriate quantity of diluents may be used because of a different vial size. This could result in a improper dose being administered to a patient; and
e) Cost of manufacture: The manufacture of a lyophilized formulation is quite costly, since it not only requires capital investment for installation of a lyophiliser, but also its maintenance;
   even though, the stability of the reconstituted solution is not a major issue, since such solutions need to be administered immediately or within a prescribed time, generally not exceeding 24 hrs.

A preformed solution of disodium pamidronate in aqueous solvents, generally referred to as “ready-to-use” solution, has found wide utility in comparison to a solution reconstituted from a lyophilizate as it overcomes the limitations associated with a lyophilized composition.

However, storage stability of such ready-to-use solutions and the tendency of disodium pamidronate solution to form particulate matter, especially when stored in glass containers over a period of time are major concerns, a problem which has vexed researchers and manufacturers all along. It is known that, the potency of the active substance in the injection solutions continuously decreases on storage for reasons unknown and also over a period of time the content of ionic metals such as aluminium, calcium, silicon etc. increases on storage rendering such solutions unsafe for human administration. The reasons for later phenomenon could be attributed to pH of the disodium pamidronate solutions, with reports suggesting the level of particulate matter content or turbidity of the solution being more pronounced with increase in pH (US 2004/0082545A 1).

The abovementioned problems have been attempted to be overcome essentially by two means, viz.
a) Adjusting the pH of the solution to near neutral or slightly acidic range; and
b) Through use of storage containers and/or elastomeric closures with a material of construction such that it does not react with disodium pamidronate solution thereby minimizing the formation of particulate matter.

To name a few:
i) The disclosure made by Shinal et. al. in U.S. Pat. No. 6,794,536 teaches an aqueous solution of disodium pamidronate having a pH of about 6.5, prepared by insitu formation of disodium pamidronate comprising the steps of addition of aqueous sodium hydroxide solution to an aqueous solution of pamidronic acid followed by adjustment of pH to 6.5 with phosphoric acid.
ii) In another disclosure made by Winter et. al. in U.S. Pat. No. 5,662,918, the problem of stability and turbidity has been claimed to be solved by providing a solution of disodium pamidronate in water having a pH of about 3.0 to 4.5, the solution in addition containing a polyethylene glycol. The acidic pH of 3.0 to 4.5 and use of polyethylene glycols is further claimed to result in an aluminium content of less than 2 ppm.
iii) In U.S. Pat. No. 6,709,674, Mirejovsky et. al. have reported use of non-glass containers for storage of aqueous composition of pamidronate disodium having a pH of 6.5. Such containers are those wherein the inner surface is made up of plastics such as polypropylene, polyethylene or polymethylpentene. The patent claims that by using such containers, the silicon content in the solution could be drastically reduced.
iv) While Handreck et. al. in published application US 2004/0,082,545 disclose a stable and clear disodium pamidronate solution having a pH of between 5.0 and 8.0, however, the said stability and clarity is supposedly achieved again through use of a glass container, the inner surface of which is pretreated with silicone oil or through use of types of glasses which have low aluminium content. The patent also recommends use of special stoppers in combination with abovementioned containers for optimum stability and clarity.
v) Similarly, Szymanski et. al. in published application US 2004/0,147,486 also recommended use of a non-reactive container, especially plastic containers for storage of disodium pamidronate solution.

From the foregoing disclosures, it would be abundantly evident that a higher pH i.e. a pH in the alkaline range is detrimental not only to storage stability but also to the clarity of a bisphosphonate salt solution, specially pamidronate disodium solution, a fact endorsed by the availability of such solutions all having a pH of about 6.5.

It would be further evident that adjustment of pH to about 6.5 alone does not suffice to solve the problem of stability and clarity. It also depends greatly on the material of construction of containers as well as the stoppers used for storage of the solution. In general, non-glass containers and coated rubber closures being more preferred.

It might be mentioned that while adjustment of pH of a solution is a simple technique, that can be practiced by any person skilled in the art and, moreover, neither involves an extra unit operation in manufacturing nor significantly adds to the cost of the manufacture, however, the same does not hold true in the case of usage of a non-glass or specially treated glass containers for storage for the reasons given herein below:

The non-glass containers, made up of plastics such as polypropylene, polyethylene or polymethylpentene, in the first place being non-biodegradable raise serious environmental concerns in their disposal. Secondly, use of such non-glass containers does not guarantee a clear, particulate free solution since in the manufacture of such containers various other ingredients such as plasticizers, lubricants, mold release agents, pigments, stabilizers, antioxidants, and binding or antistatic agents, are utilized to perform a specific function during fabrication, which like aluminium, calcium, silicon can be leached into the solution on storage. Finally, certain ingredients of the drug preparation may bind to the plastic or be absorbed by it, and oxidation, degradation, or precipitation of the drug product may occur. It is also possible for a component of the drug product to migrate through the walls of the container, and oxygen, carbon dioxide, or other gases may pass through the plastic into the drug system. Moreover, use of a plastic container does not allow the administrator to inspect the vial prior to infusion, for the presence of particulate matters, which is normally recommended by Health Authorities to ensure the absence of any visible extraneous matter in the solutions especially the ones to be administered intravenously and thus ensure safety of the solutions, being administered to a patient.

With regard to pre-treated glass containers or glass containers wherein the inner surface is made non-reactive, it should be noted that their manufacture involves complex technology. While it is outside the scope of this specification to discuss the technology involved in manufacture of such types of glass containers, however their cost would be a measure of the technology utilized. For instance, the cost of glass vials pretreated with silicone oil is approximately Euro 2200 per 1000 vials which is nearly twenty times that of normal Type I or untreated glass vials, which cost approximately Euro 120 per 1000 vials. Obviously, a disodium pamidronate pharmaceutical composition prepared and stored in a pre-treated glass container would be available to patients at a considerably higher cost rendering it to be a privy of the wealthy and not the needy.

Similarly, a rubber closure is an essential part of packaging systems used for parenterals. The routinely used elastomeric closures are selected from butyl rubber, halobutyl rubber, ethylenepropylene rubber, silicone rubber, fluoroelastomers, neoprene rubber, styrene butadiene rubber and polybutadiene rubber closures. Even coating of rubber closure with fluorinated resins such as tetrafluoroethylene polymer, trifluorochloroethylene polymer, fluorovinylidene polymer, perfluoroalkoxy polymer etc. is used to form an inert barrier. A cost analysis of these rubber closures indicate that coated rubber closures are at least two times costlier than the normal halobutyl rubber closures. Therefore, use of such specially treated rubber closure again will add to the cost of the pharmaceutical compositions making them available to patients at considerably higher cost.

A great need, if not imperative, exists for a pharmaceutical composition of disodium pamidronate, which is not only simple and convenient, but more importantly is available to the public at large at a reasonable cost.

### OBJECTS OF THE INVENTION

An object of the present invention is to provide a pharmaceutical composition of disodium pamidronate, which is storage stable.

Another object of the present invention is to provide a pharmaceutical composition of disodium pamidronate, which is stable inconventional glass containers.

Still another object of the present invention is to provide a pharmaceutical composition of disodium pamidronate, which has an alkaline pH.

A further object of the present invention is to provide a pharmaceutical composition of disodium pamidronate, which is devoid of any turbidity on storage for pharmaceutically acceptable duration of time.

Another object of the present invention is to provide a process for preparation of a stable pharmaceutical composition of disodium pamidronate, which is simple, convenient and economical.

Yet another object of the present invention is to provide a method for inhibition of bone resorption of a human or an animal cancerous disease.

Contrary to the teachings of the prior art, the present inventors have found to their surprise that indeed a solution of disodium pamidronate having an alkaline pH and stored in a conventional and untreated glass container and rubber closure system is not only stable but also is substantially free of particulate matters when stored for a pharmaceutically acceptable duration of time.

In particular, for obtaining a stable and particulate matter free solution of disodium pamidronate it has been found that recourse to neither adjustment of pH nor utilization of non-glass or especially treated glass containers are required and pharmacopoeially and pharmaceutically acceptable stability and clarity could be achieved through a simple and significantly less expensive unit operation of subjecting the said solution to steam sterilization.

In addition, it has been also found that recourse to utilization of stoppers of special make or to say in other words, coated stoppers, need not be taken for achieving pharmacopoeially and pharmaceutically acceptable stability and clarity of the disodium pamidronate solution. Incidentally, halobutyl rubber could be employed to obtain a stable and particulate matter free solution of disodium pamidronate.

### SUMMARY OF THE INVENTION

Thus the present invention relates to a stable pharmaceutical composition comprising a solution of disodium pamidronate in an aqueous solvent.

The solution of disodium pamidronate according to the invention is free of particulate matter. The aqueous solvent having an alkaline pH, preferably a pH from about 8.0 to 8.5.

According to an aspect of the invention the composition comprises a sugar.

The aqueous solvent according to the invention is filled and stored in a conventional and untreated glass container and sealed with normal elastomeric closure system.

In still another aspect, the present invention provides a process for preparation of the stable pharmaceutical composition according to the invention comprising the steps of:
i) adding disodium pamidronate and optionally a sugar into an aqueous solvent;
ii) heating the mixture of step (i) at a temperature in the range of 50° C. to 90° C. to obtain a clear solution.

The clear solution is cooled to ambient temperature and filtered. The filtered solution is filled into a conventional, untreated glass container and sealed with an elastomeric closure. The sealed container is sterilized by steam sterilization.

### DETAILED DESCRIPTION OF THE INVENTION

The pharmaceutical composition of the present invention comprises of a solution of disodium pamidronate in an aqueous solvent.

In particular, as mentioned hereinbefore, the pharmaceutical composition of the present invention comprises of a solution of disodium pamidronate and a sugar in an aqueous solvent having a pH from about 8.0 to 8.5 wherein the solution is filled into primary packaging of USP Type I glass which is then terminally sterilized with steam sterilization.

As used herein the term “aqueous solvent” refers to water containing solvents. Pure sterile water for injection is preferred. Mixtures of water and one or more auxiliary carriers or co-solvents like ethanol, benzyl benzoate or mixtures thereof can be employed.

The final water content in the solution of disodium pamidronate ranges from 0.01 to 99.99%.

Sugars, which may be used in the pamidronate disodium composition of the present invention, include mannitol, lactose and sucrose. Typically the sugar is mannitol.

The elastomeric closure system is a halobutyl rubber closure system. The halobutyl rubber closure is selected from chlorobutyl and bromobutyl rubber closure.

The glass container is a USP Type I hydrolytic glass container. As used herein the term “conventional and untreated glass” refers to USP Type I glass, commonly known as “normal hydrolytic class-I glass” or borosilicate glass as classified by United States Pharmacopoeia. Example of such glasses are, but not limited to, Corning® Pyrex® 7740 and Wheaton 180, 200, and 400.

Further, both molded and tubular USP Type I glasses could be employed in the present invention.

The primary packaging material which could be employed in the present invention include, but is not limited to, ampoules, vials, ready-to-use syringes, carpoules etc.

Steam sterilization of a test composition comprises heating the said test composition at a temperature of about 120° C. to 150° C. for a period of 15-120 minutes at a pressure of about 1 bar or more. By test composition, it means a solution of disodium pamidronate in an aqueous solvent, optionally containing a sugar, and having an alkaline pH, sealed in a conventional, untreated glass container and moreover having normal halobutyl rubber closures.

Preferably, the sterilization is carried out at a temperature in the range of about 120° C. to 140° C. and more preferably at a temperature of 120° C. to 125° C. While the sterilization can be carried out for a period of 15-120 minutes, however, it is preferable to carry out the same for a period of 15-60 minutes and more preferably, to carry out for a period of 15-20 minutes. Typically, the pressure applied is in the range of 1-20 bar and more preferably in the range of 1-5.

The amounts of disodium pamidronate used in the formulation according to present invention vary from about 0.1 mg/ml to 100.0 mg/ml. The amount, which is present, is not critical and may be adjusted in accordance with the individual needs and preferences.

Typically, the concentration of disodium pamidronate will be about 3.0 mg/ml to 9.0 mg/ml.

The stable pharmaceutical composition of the present invention could be prepared by a process, which comprises of the following steps:
i) Addition of Disodium Pamidronate and optionally a sugar into an aqueous solvent;
ii) Heating the mixture of step (i) at a temperature in the range of 50° C. to 90° C. to obtain a clear solution;
iii) Cooling the solution of step (ii) to ambient temperature;
iv) Filtering the solution of step (iii);
v) Filling the clear solution of step (iv) into a conventional, untreated glass container;
vi) Sealing the container as prepared in step (v) with an elastomeric closure and
vii) Sterilizating the sealed container of step (vi) by steam sterilization.

Sterilization of an injectable solution can be carried out through various physical and chemical methods known in the art. The physical methods, most widely utilized to sterilize parenteral solutions, are filtration, radiation and steam sterilization. However, in the present context, it was found that in particular sterilization of an aqueous solution of disodium pamidronate having an alkaline pH in the range of 8.0 to 8.5 by steam sterilization method was vastly superior over the other methods in minimization of particulate matter formation.

As a point of illustration, a comparison of the particulate matter content of an aqueous solution of disodium pamidronate having a pH of around 8.0 to 8.5, filled and stored in a conventional USP Type I glass vial, both molded and tubular with halobutyl rubber closure, and sterilized through aseptic processing and steam sterilization is summarized in Table-I.

TABLE I
Comparison of the Stability and Particulate Matter Content of an Aqueous
Solution of Disodium Pamidronate having a pH of around 8.0 to 8.5 Sterilized
through Aseptic Processing and steam sterilization methods.

| | | | Stability | Particulate matter | | Particulate matter | |
|---|---|---|---|---|---|---|---|
| Strength | Sterilization | Storage | Duration | (Molded Vials) | | (Tubular Vials) | |
| (mg/ml) | Method | conditions | (months) | >10 μm | >25 μm | >10 μm | >25 μm |
| 3.0 | Aseptic processing | 25° C. | 9 | 811.67 | 0.00 | 1111.67 | 36.67 |
| | Steam sterilization | 25° C. | 10 | 26.67 | 1.67 | 48.33 | 1.67 |
| 9.0 | Aseptic processing | 25° C. | 9 | 5546.67 | 966.67 | 5340.00 | 873.33 |
| | Steam sterilization | 25° C. | 10 | 88.33 | 13.33 | 70.00 | 6.67 |

From Table-I, it would be abundantly evident that there is a many fold increase of particulate matter content in the disodium pamidronate composition sterilized by aseptic processing over that obtained by steam sterilization.

Further, steam sterilization method is not only simple and requires relatively short processing time but is also more cost effective since unlike aseptic processing, no capital expenditure is incurred for installation of microbiological filters and cleanroom environments.

The pharmaceutical compositions of disodium pamidronate are highly effective for treatment of bone pain associated with tumors, in both human and animal hosts, such as in moderate or severe hypercalcemia of malignancy, Paget's disease of bone, osteolytic bone lesions of multiple myeloma, and osteolytic bone metastases of breast cancer. Pamidronate may also be effective in treating bone pain associated with other tumors like prostate cancer. Disodium pamidronate is also useful in treating bone marrow edema syndrome or transient osteoporosis of the hip, fibrous dysplasia, and melorheostosis.

The pharmaceutical compositions can be administered by single dose intravenous infusion of 60 to 90 mg over at least 2 to 24 hours.

The pharmaceutical compositions thus prepared exhibit excellent storage stability as would be evident from the examples given herein below, which are not limiting and should not be construed as limiting the scope of the invention.

### EXAMPLE-1

To compare the effect of sterilization process (steam sterilization vs aseptic processing) and nature of container and closure on the stability of aqueous solution of pamidronate disodium, the solution of disodium pamidronate having a pH of around 8.2 and having the following composition was prepared.

| | |
|---|---|
| Disodium Pamidronate | 9 mg/ml |
| Mannitol | 37.5 mg/ml |
| Water for Injection | q.s. to 10 ml |

The abovementioned solution was divided into four equal parts. The first two parts were aseptically processed in to 10 ml, USP Type-I, tubular as well as molded glass vials and stoppered with two different types of 20 mm stoppers. The remaining two parts were filled in to 10 ml, USP Type-1, tubular as well as molded glass vials and stoppered with two different types of 20 mm stoppers. These vials were then sterilized terminally by autoclaving at 120° C. for 20 minutes.

The vials were subjected to stability testing in upright as well as inverted position at 25° C./60% RH and 50° C. and observed for 12 months and 3 months respectively. Their stability was evaluated with respect to the clarity of solution and appearance of particulate matter. The obtained results are summarized in Table-II.

TABLE II
Stability Studies of Disodium Pamidronate Solutions.

| | | | Stability durations (months) | | | |
|---|---|---|---|---|---|---|
| | | | 25° C./60% RH | | 50° C. | |
| Sterilization | | | Upright | Inverted | Upright | Inverted |
| method | Nature of vials | Nature of rubber closure | position | position | position | position |
| Aseptic | Molded | 4405/50 Bromobutyl | <6 | <6 | <3 | <3 |
| processing | | D777-1, Flurotec | <6 | <6 | <3 | <3 |
| | Tubular | 4405/50 Bromobutyl | <6 | <6 | <3 | <3 |
| | | D777-1, Flurotec | <6 | <6 | <3 | <3 |
| Autoclaving | Molded | 4405/50 Bromobutyl | >12 | >12 | >3 | >3 |
| | | D777-1, Flurotec | >12 | >12 | >3 | >3 |
| | Tubular | 4405/50 Bromobutyl | >12 | >12 | >3 | >3 |
| | | D777-1, Flurotec | >12 | >12 | >3 | >3 |

No significant difference was observed in stability for upright and inverted positioned disodium pamidronate vials. Further, the Flurotec stoppers were also found to be equivalent to the Bromobutyl stopper. Even further, no significant difference was observed between molded and tubular vials indicating superior stability of the product in autoclaved vials.

### EXAMPLE-2

This example illustrates the physico-chemical properties of pamidronate solution prepared in accordance with present invention. Aqueous solutions of pamidronate disodium having a pH of around 8.2 and having the following composition were prepared.

| | | |
|---|---|---|
| Disodium Pamidronate | 9 mg/ml | 3 mg/ml |
| Mannitol | 37.5 mg/ml | 47.0 mg/ml |
| Water for Injection | q.s. to 10 ml | q.s. to 10 ml |

The solutions were filled in to 10 ml, USP Type-I, clear, colorless tubular glass vials and stoppered with 20 mm 4405/50 Bromobutyl stoppers. The vials were then sterilized terminally by autoclaving and were kept upright and inverted at 25° C./60% RH and 40° C./75% RH. The analysis of the physico-chemical properties of the pamidronate solutions at various time periods is set forth in Table-III.

TABLE III
Physical Properties of the Pamidronate Disodium solutions prepared in accordance with the present disclosure

| | | Storage conditions | | | | | | | | | | | | | |
|---|---|---|---|---|---|---|---|---|---|---|---|---|---|---|---|
| | | 25° C./60% RH | | | | | | 40° C./75% RH | | 50° C. | | | | | |
| | | 1 M | | 2 M | | 6 M | | 6 M | | 1 M | | 2 M | | 3 M | |
| Test | Initial | ↑ | ↓ | ↑ | ↓ | ↑ | ↓ | ↑ | ↓ | ↑ | ↓ | ↑ | ↓ | ↑ | ↓ |

3 mg/ml solution

| | | | | | | | | | | | | | | | |
|---|---|---|---|---|---|---|---|---|---|---|---|---|---|---|---|
| Visual color | CL | CL | CL | CL | CL | CL | CL | CL | CL | CL | CL | CL | CL | CL | CL |
| Visual clarity | clear | clear | clear | clear | clear | clear | clear | clear | clear | clear | clear | clear | clear | clear | clear |
| pH | 8.1 | 8.1 | 8.1 | 8.1 | 8.2 | 8.17 | 8.21 | 7.89 | 8.05 | 8.1 | 8.0 | 8.0 | 8.0 | 8.0 | 8.0 |
| Assay (mg/ml) | 3.06 | 3.07 | 3.11 | 2.97 | 2.99 | 3.0 | 2.99 | 3.03 | 2.97 | 3.11 | 3.06 | 3.02 | 3.03 | 2.99 | 3.03 |
| % β Alanine | <0.05 | <0.05 | <0.05 | <0.05 | <0.05 | <0.05 | <0.05 | <0.05 | <0.05 | <0.05 | <0.05 | <0.05 | <0.05 | <0.05 | <0.05 |
| % Phosphate | <0.05 | <0.05 | <0.05 | <0.05 | <0.05 | <0.05 | <0.05 | <0.05 | <0.05 | <0.05 | <0.05 | <0.05 | <0.05 | <0.05 | <0.05 |
| % Phosphite | <0.05 | <0.05 | <0.05 | <0.05 | <0.05 | <0.05 | <0.05 | <0.05 | <0.05 | <0.05 | <0.05 | <0.05 | <0.05 | <0.05 | <0.05 |

9-mg/ml solution

| | | | | | | | | | | | | | | | |
|---|---|---|---|---|---|---|---|---|---|---|---|---|---|---|---|
| Visual color | CL | CL | CL | CL | CL | CL | CL | CL | CL | CL | CL | CL | CL | CL | CL |
| Visual clarity | clear | clear | clear | clear | clear | clear | clear | clear | clear | clear | clear | clear | clear | clear | clear |
| pH | 8.1 | 8.3 | 8.3 | 8.2 | 8.2 | 8.19 | 8.25 | 8.18 | 8.18 | 8.2 | 8.2 | 8.1 | 8.1 | 8.2 | 8.2 |
| Assay (mg/ml) | 9.18 | 9.17 | 9.17 | 9.14 | 9.22 | 9.13 | 9.05 | 9.14 | 9.12 | 9.23 | 9.10 | 8.92 | 9.14 | 9.01 | 9.07 |
| % β Alanine | <0.05 | <0.05 | <0.05 | <0.05 | <0.05 | <0.05 | <0.05 | <0.05 | <0.05 | <0.05 | <0.05 | <0.05 | <0.05 | <0.05 | <0.05 |
| % Phosphate | <0.05 | <0.05 | <0.05 | <0.05 | <0.05 | <0.05 | <0.05 | <0.05 | <0.05 | <0.05 | <0.05 | <0.05 | <0.05 | <0.05 | <0.05 |
| % Phosphite | <0.05 | <0.05 | <0.05 | <0.05 | <0.05 | <0.05 | <0.05 | <0.05 | <0.05 | <0.05 | <0.05 | <0.05 | <0.05 | <0.05 | <0.05 |

↑ = stored upright,
↓ = stored inverted,
CL = colourless,
RH = relative humidity,
M = month

The pamidronate solutions of the present invention exhibited good long term storage stability. The pamidronate solutions retained their potency and clarity. Moreover, pamidronate solutions did not exhibit degradation as evidenced by the negligible levels of the impurities present.

### EXAMPLE-3

This example illustrates the number counts of sub-visible particulate matter present in the disodium pamidronate solution prepared in accordance with present invention and its comparison with that of marketed formulations.

The disodium pamidronate solutions of strength 3 mg/ml and 9 mg/ml were prepared in a manner similar to that described in Examples 1 and 2. Pamidronate disodium samples available from the market belonging to M's Novartis (Aredia®), M's Bedford and M/s Faulding were also evaluated for number counts of sub-visible particulate matter and obtained results are presented in Table-IV.

TABLE IV
Comparison of Sub-visible Particulate Matter counts
in different Disodium Pamidronate Solutions

| | | | Sub-visible | |
|---|---|---|---|---|
| Storage | | | particles | |
| conditions | Product | Strength | >10 μm | >25 μm |
| 25° C./60% | Solution according to | 3 mg/ml | 26.67 | 1.67 |
| RH - 10 months | present invention - | | | |
| | Molded vials | | | |
| 25° C./60% | Solution according to | 3 mg/ml | 48.33 | 1.67 |
| RH - 10 months | present invention - | | | |
| | Tubular vials | | | |
| 25° C. | Aredia ®^{@} | 3 mg/ml | 566.67 | 0.00 |
| | (Reconstituted with WFI) | | | |
| 25° C. | Bedford^{#} | 3 mg/ml | 30.00 | 1.67 |
| | (Ready-to-use solution) | | | |
| 25° C./60% | Solution according to | 9 mg/ml | 88.33 | 13.33 |
| RH - 10 months | present invention - | | | |
| | Molded vials | | | |
| 25° C./60% | Solution according to | 9 mg/ml | 70.00 | 6.67 |
| RH - 10 months | present invention - | | | |
| | Tubular vials | | | |
| 25° C. | Aredia ®* | 9 mg/ml | 480.00 | 0.00 |
| | (Reconstituted with WFI) | | | |
| 25° C. | Faulding⁺ | 9 mg/ml | 38.33 | 0.00 |
| | (Ready-to-use solution) | | | |
| 25° C. | Faulding⁺⁺ | 9 mg/ml | 45.00 | 1.67 |
| | (Ready-to-use solution) | | | |

^{@}= Batch Number 193G1993, Expiry - March 2005 (freshly reconstituted),
*= Batch Number 221E3280, Expiry - October 2004 (freshly reconstituted),
^{#}= Batch Number US 342785, Expiry - September 2005,
⁺= Batch Number UK N124947, Expiry - June 2005,
⁺⁺= Batch Number US 03P602, Expiry - November 2005.
RH = Relative Humidity

### EXAMPLE-4

This example illustrates the number of metallic ions present in the 9 mg/ml disodium pamidronate solution prepared in accordance with present invention. Table-V shows the test results measured over a period of 3 months while being stored at 25° C./60% relative humidity (RH) and 40° C./75% relative humidity and 50° C.

TABLE V
Analysis of Metallic Ions Present in
the Disodium Pamidronate Formulations

| | Calcium | Barium | Aluminium | Silicates |
|---|---|---|---|---|
| Storage conditions | (ppm) | (ppm) | (ppm) | (ppm) |
| Initial | 9.36 | 0.99 | 0.97 | 10.80 |
| 25° C./60% RH/ | 5.85 | 1.43 | 2.95 | 35.59 |
| 3 months, ↑ | | | | |
| 25° C./60% RH/ | 6.22 | 2.09 | 4.33 | 35.75 |
| 3 months, ↓ | | | | |
| 40° C./75% RH/ | 7.06 | 2.94 | 5.38 | 69.4 |
| 3 months, ↑ | | | | |
| 40° C./75% RH/ | 7.43 | 3.47 | 6.57 | 72.0 |
| 3 months, ↓ | | | | |
| 50° C./1 month, ↑ | 7.20 | 2.40 | 4.4 | 45.4 |
| 50° C./1 month, ↓ | 7.80 | 3.50 | 4.0 | 31.3 |

↑ = stored upright,
↓ = stored inverted.

No significant difference was observed in metal ions content for upright and inverted positioned disodium pamidronate vials of strength 9 mg/ml. The level of calcium ions is nearly constant over a period of 3 months when stored at various temperature conditions. The level of other metal ions like aluminium, barium and silicates showed a non significant increase of 1.5 to 6 folds over initial values, when stored at various temperature conditions for a duration of 3 months, when compared to the reported values for metal ions in pamidronate solution. For instance, disodium pamidronate solutions reported in U.S. Pat. No. 5,662,918 showed a 6 to 7 fold increase when stored for 13 weeks. Moreover, strength of this solution was 1.12 mg/ml.

It is understood that various modifications, alterations and/or addition may be made to the product specifically described herein without departing from the spirit and ambit of the invention.

1. A stable pharmaceutical composition comprising disodium pamidronate in an aqueous solvent having an alkaline pH of above 8.0, filled and stored in a conventional, untreated glass container, wherein the aqueous solvent is in contact with a glass surface of the container.

2. A stable pharmaceutical composition comprising disodium pamidronate in an aqueous solvent having an alkaline pH of above 8.0, filled and stored in a conventional, untreated glass container and sled with an elastomeric closure, wherein the aqueous solvent is in contact with a glass surface of the container.

3-4. (canceled)

5. A stable pharmaceutical composition according to claim 1, further comprising a sugar.

6. A pharmaceutical composition according to claim 1, wherein die conventional, untreated glass container is a USP Type I glass container.

7. A pharmaceutical composition according to claim 30, wherein the conventional, untreated glass container is selected from ampoules, vials, ready-to-use syringes and carpoules.

8. A pharmaceutical composition according to claim 42 wherein the elastomeric rubber closure is a halobutyl rubber closure.

9. A pharmaceutical composition according to claim 8, wherein the halobutyl rubber closure is selected from chlorobutyl and bromobutyl rubber closure.

10. A pharmaceutical composition according to claim 5, wherein the sugar is selected from dextrose, lactose and mannitol.

11. A pharmaceutical composition according to claim 10, wherein the sugar is mannitol.

12. A pharmaceutical composition according to claim 1, wherein the pH of the solution is from about 8.0 to 8.5.

13. A pharmaceutical composition according to claim 12, wherein the pH of the solution is from about 8.0 to 8.2.

14. A pharmaceutical composition according to claim 1, wherein the aqueous solvent is selected from water, ethanol, benzyl benzoate or mixtures thereof.

15. A pharmaceutical composition according to claim 14, wherein the aqueous solvent is water.

16. A stable pharmaceutical composition according to claim 15, wherein the final water content in the solution of disodium pamidronate ranges from 0.01 to 99.99%.

17. A process for preparation of a pharmaceutical composition according to claim 1 comprising the steps of:
i) adding disodium pamidronate and optionally a sugar into the aqueous solvent;
ii) heating the mixture of step (i) at a temperature in the range of 50° C. to 90° C. to obtain a clear solution,
wherein the solution has a pH above 8.0.

18. A process according to claim 17, wherein the clear solution of step (ii) is cooled to ambient temperature and filtered.

19. A process according to claim 18 wherein the filtered solution is filled into the conventional, untreated glass container and sealed.

20. A process according to claim 19 wherein the sealing is made with an elastomeric closure.

21. A process according to claim 20 wherein the sealed container is sterilized by steam sterilization.

22. A process according to claim 17, wherein the aqueous solvent is selected from water, ethanol, benzyl benzoate or mixtures thereof.

23. A process according to claim 22, wherein the aqueous solvent is water.

24. A process according to claim 23, wherein the sugar is selected from dextrose, lactose and manitol.

25. A process according to claim 24, wherein the sugar is mannitol.

26. A process according to claim 17, wherein the conventional, untreated glass container is a USP type I glass container.

27. A process according to claim 17, wherein the elastomeric closure is a halobutyl rubber closure.

28. A process according to claim 17, wherein the sterilization is achieved through autoclaving.

29. A process according to claim 34, wherein autoclaving is performed at a pressure ranging from about 1 to 5 bar.

30. A pharmaceutical composition according to claim 6, wherein the conventional, untreated glass container is tubular or molded.

31. A process according to claim 26, wherein the conventional, untreated glass container is tubular or molded.

32. A process according to claim 26, wherein the conventional, untreated glass container is selected from ampoules, vials, ready-to-use syringes and carpoules.

33. A process according to claim 27, wherein the halobutyl rubber closure is selected from bromobutyl and chlorobutyl rubber closures.

34. A process according to claim 28, wherein the autoclaving is performed at a temperature ranging from about 120° C. to 150° C.; for a time duration ranging from about 15 to 120 minutes; and at a pressure ranging from about 1 to 20 bar.

35. A process according to claim 34, wherein autoclaving is performed at a temperature of about 120° C. to 125° C.

36. A process according to claim 35, wherein autoclaving is performed for at least 15 minutes.
